(19) United States
(12) Patent Application Publication
(10) Pub. No.: US 2009/0192340 A1
(43) Pub. Date: Jul. 30, 2009

(54) ALKYLAROMATIC DEHYDROGENATION SYSTEM AND METHOD FOR MONITORING AND CONTROLLING THE SYSTEM

(76) Inventors: Robert Dielman CULP, Katy, TX (US); Lorna Beatriz ORTIZ-SOTO, Katy, TX (US); John David WEBB, Hayden, ID (US)

Correspondence Address: SHELL OIL COMPANY, P O BOX 2463, HOUSTON, TX, 772522463, US

(21) Appl. No.: 12/262,049

(22) Filed: Oct. 30, 2008

Related U.S. Application Data
(60) Provisional application No. 60/984,669, filed on Nov. 1, 2007.

(51) Int. Cl. C07C 5/333; B01D 19/00; G01J 5/02

(52) U.S. Cl. 585/440; 422/119; 250/340

(57) ABSTRACT

An alkylaromatic dehydrogenation system is described. In addition, a method is described for monitoring an alkylaromatic dehydrogenation process comprising: drawing a sample from the process at a first sample point; passing the sample through an analyzer; and measuring the amount of at least one component present in the sample wherein the sample is at least partially uncondensed. An apparatus is also described for monitoring an ethylbenzene dehydrogenation process comprising a plurality of sample lines that are heat-traced sufficiently to inhibit condensation in the sample lines; and a Fourier Transform Infrared Spectrometer comprising two sample cells wherein the ratio of the length of a first sample cell to the length of the second sample cell is from about 1:1000 to about 1:1.

### CROSS REFERENCE TO RELATED APPLICATION

This application claims priority to U.S. Provisional Application Ser. No. 60/984,669 filed Nov. 1, 2007, the entire disclosure of which is herein incorporated by reference.

### FIELD OF THE INVENTION

This invention relates to an alkylaromatic dehydrogenation process and a method of monitoring and controlling the dehydrogenation process.

### BACKGROUND OF THE INVENTION

The importance of accurately monitoring chemical process units is recognized by the industry, but there are not always effective methods to monitor all aspects of certain processes. For example, the current practice for monitoring an alkylaromatic dehydrogenation process is to extract samples from the reactor or effluent stream, condense the sample and measure the components using a gas chromatograph. This manual sampling is time consuming and requires manpower to carry out each step. Due to the burden of sampling, it is usually only done once a day or less frequently so it does not provide timely information to allow the reaction conditions to be changed if necessary. In addition, the precision and accuracy of the results depends greatly on the technique of the operator, temperature of the condensation, and thoroughness of the capture of condensable liquids. The sampling lines often become plugged due to condensation and polymerization in the lines making it even more difficult to take the samples. In addition, the conventional method of sampling, condensing the sample and using a gas chromatograph to measure the components does not allow for all of the components to be measured. It is difficult to measure the components such as alpha-methylstyrene, phenylacetylene, cumene, divinylbenzene, xylenes and other heavies that are only present in very small amounts and it is not possible to measure the quantity of uncondensable gases, for example carbon monoxide and carbon dioxide.

U.S. Pat. No. 5,684,580 describes the use of a Raman spectrometer for monitoring the product composition of a purified styrene monomer stream produced by an ethylbenzene dehydrogenation process. The patent also provides for the control of the dehydrogenator, the distillation columns, and/or the amount of polymerization inhibitor addition. The patent describes measuring the amount of unconverted ethylbenzene in the purified styrene monomer stream after the stream has been separated in a series of separation vessels, so it does not provide an analysis of all the components, especially the light and uncondensable components, exiting the reactor. The patent describes measuring a liquid styrene product stream and this requires the addition of a polymerization inhibitor to prevent plugging in the lines.

### SUMMARY OF THE INVENTION

The invention provides a method for monitoring an alkylaromatic dehydrogenation process comprising drawing a sample from the process at a first sample point, passing the sample through an analyzer and measuring the amount of at least one component present in the sample wherein the sample is at least partially uncondensed.

The invention further provides an alkylaromatic dehydrogenation system comprising: a dehydrogenation reaction zone comprising a dehydrogenation catalyst and having a plurality of sample points along the length of the reaction zone; a plurality of sample lines providing a flow path from the sample points to an infrared analyzer wherein the sample lines are protected from heat loss sufficiently to inhibit condensation in the lines.

The invention provides a method for controlling the reaction conditions of an alkylaromatic dehydrogenation system comprising contacting a feed comprising an alkylaromatic compound with a dehydrogenation catalyst; drawing a sample from the dehydrogenation system; passing the sample through an analyzer comprising a Fourier Transform Infrared Spectrometer; measuring the amount of components in the sample; and adjusting the reaction conditions to improve dehydrogenation system performance wherein the sample is at least partially uncondensed when it is passed through the analyzer.

The invention further provides an apparatus for monitoring an ethylbenzene dehydrogenation process comprising a plurality of sample lines that are heat-traced sufficiently to inhibit condensation in the sample lines; and a Fourier Transform Infrared Spectrometer comprising two sample cells wherein the ratio of the length of a first sample cell to the length of the second sample cell is from about 1:1 to about 1:1000

### BRIEF DESCRIPTION OF THE DRAWINGS

FIG. 1 depicts an embodiment of an alkylaromatic dehydrogenation system.

FIG. 2 depicts an embodiment of an alkylaromatic dehydrogenation system comprising an oxidative reheating reactor.

### DETAILED DESCRIPTION OF THE INVENTION

The invention provides a method for monitoring a dehydrogenation process that alleviates the burdens associated with conventional manual sampling and measuring the components using a gas chromatograph. The conventional method requires considerable time and labor, so samples are usually drawn and tested on a daily basis. This does not provide adequate data to improve the efficiency of the operation of the alkylaromatic dehydrogenation process.

This method also provides for measuring components that cannot be measured using the conventional methods. Many components are present in the dehydrogenation process at very low levels, measured in parts per million, and can not be adequately measured using the conventional methods that require condensation of the sample before analysis. This invention provides a method that allows these components to be measured. The components that are present at very low levels include alpha-methylstyrene, phenylacetylene, cumene, divinylbenzene, xylenes, indene, benzaldehyde, diethylbenzene, propylbenzene, stilbene, non aromatic hydrocarbons having from 5 to 10 carbon atoms, o,m,p-methylstyrene, ethyltoluene, triethylbenzene, diphenylmethane, diphenylethane, formaldehyde, and formic acid. In addition, the conventional methods are not suited to measuring the amounts of uncondensable gases, for example carbon monoxide and carbon dioxide, but the method according to this invention does provide a method of measuring uncondensable gases. There are several types of alkylaromatic dehydrogenation processes that would be benefited by this invention, but this description will describe a specific embodiment of the dehydrogenation of ethylbenzene to form styrene. One of ordinary skill in the art could apply this invention to many other similar dehydrogenation processes.

An alkylaromatic dehydrogenation system typically comprises one or more reactors loaded with dehydrogenation catalyst. The catalyst may be supported or unsupported, and is preferably an iron oxide based catalyst comprising additional components, for example, alkali metals, alkaline earth metals and catalyst promoters. Typical catalysts are described in U.S. Patent Application Publication No. 2003/0144566 herein incorporated by reference.

A typical feed to an alkylaromatic dehydrogenation system comprises a dehydrogenatable hydrocarbon, for example ethylbenzene, and steam. The steam may be introduced at a steam to hydrocarbon molar ratio of from 1 to 20, preferably from 5 to 10.

An alkylaromatic dehydrogenation system is typically operated at from 500° C. to 700° C. The absolute pressure is typically in the range of from 10 to 300 kPa, more typically from 20 to 200 kPa, for example 50 kPa, or 120 kPa. A dehydrogenation system is preferably operated at a pressure below atmospheric pressure.

The dehydrogenation process is endothermic which means that when multiple reactors are present, the feed will typically be reheated before entering each dehydrogenation reactor to maintain a reaction temperature that provides optimal performance. The heat can be added by adding additional steam or otherwise heating the effluent from one reactor before it enters the next reactor. In another embodiment, the heat may be added in the reactor. In one embodiment, heat may be added to tubular reactors through the walls of the reactor tubes provided by a hot fluid or an electric furnace.

In some embodiments, the dehydrogenation process comprises an oxidative reheating reaction zone that is used to combust hydrogen present in the process to reheat the stream and improve the conversion by shifting the equilibrium as described in U.S. Pat. No. 5,043,500 hereby incorporated by reference. The combustion occurring in this oxidative reheating reaction zone produces carbon monoxide and carbon dioxide, which can have a negative impact on the activity of the catalyst in subsequent dehydrogenation reactors. The conventional method does not provide a way to monitor the levels of these gases, so this invention provides an improved method of monitoring a dehydrogenation process comprising an oxidative reheating reaction zone.

To measure the components present in the dehydrogenation system, samples must be drawn from the system and then passed to an analyzer. This sampling system may comprise one or more sample points located on the reactor and/or one or more sample points located on the feed or effluent lines connected to the reactors. In a preferred embodiment, multiple sample points are located on the reactor itself. The number of sample points will depend on the design of the reactor and the amount of information desired. For example, a pilot plant reactor in a laboratory setting may have from 3 to 10 sample points to provide suitable data for research and testing purposes.

In general, a vacuum pump will be used to draw samples from the sample points and pass them to the analyzer, although other methods are available for passing the sample to the analyzer.

One of ordinary skill in the art can design an appropriate sampling system with the necessary valves and lines to pass the sample stream from the sample point to the analyzer. The lines are preferably insulated and/or heat-traced to prevent cooling of the stream in the lines, which can result in condensation and polymerization. The stream will be at least partially uncondensed when it is passed through the analyzer. At least 80 wt % of the sample stream is present in a vapor phase, more preferably at least 90 wt %, and most preferably at least 95 wt % of the sample stream is present in a vapor phase. The stream may be completely in a vapor phase.

An inert gas purge may be used to clear the lines that are not being used to pass sample streams to the analyzer. In one embodiment, a sample from a first sample point will be passed to the analyzer for a specified amount of time. After that amount of time, the appropriate valves will operate to pass a sample stream from a second sample point to the analyzer. The portion of the line not being used while the second sample is drawn can be cleared with a flow of nitrogen or other inert gas.

When multiple sample points are used, it is preferable to sample first from the sample point that is farthest downstream from the reactor feed. In an embodiment where the reactor has four sample points, a sample will be drawn from a first sample point. Subsequently a sample will be drawn from a second sample point that is upstream of the first sample point. Subsequently a sample will be drawn from a third sample point that is upstream of the second sample point and so forth. Drawing the sample streams in this order prevents the drawing of a sample from affecting the dynamics of the reactor further downstream, which could negatively impact the accuracy of the sample analysis for the downstream sample point(s) or disturb the process.

The analyzer is preferably an infrared analyzer and more preferably an infrared analyzer comprising a Fourier Transform Infrared Spectrometer. The analyzer comprises one or more sample cells through which the samples are passed. A source of infrared radiation and a means for detecting the infrared radiation that passes through the cell are present. By determining the radiation that was absorbed by the sample stream, the quantity of each component can be determined using reference spectra of the components to be analyzed at the operation conditions of the sample cell. Due to the vacuum conditions and the high density of some of the compounds in the sample stream such as styrene and ethylbenzene it was necessary to create a set of reference spectra specific to this application. Reference spectra can typically be obtained experimentally by one skilled in the art from calibrated standards or from existing spectral libraries. The reference spectra for this application were not available in any existing spectral library.

In a preferred embodiment, the infrared analyzer comprises two sample cells of different lengths. One sample cell can be used to determine the quantity of components that are present at a level that can be measured as weight percent. Another sample cell can be used to determine the quantity of components that are present at a level that can be measured as parts per million. The use of two sample cells provides for the measurement of quantity of components that were previously not measurable using conventional techniques.

The two sample cells in the infrared analyzer are different lengths. The length of the first cell is from 5 centimeters to 20 centimeters, preferably from 8 centimeters to 12 centimeters and most preferably from 9 to 11 centimeters. The length of the second sample cell is from 5 meters to 20 meters, preferably from 8 meters to 12 meters, and most preferably from 9 to 11 meters. The ratio of the length of the first sample cell to the length of the second sample cell is from 1:1000 to 1:1, preferably from 1:400 to 1:25, more preferably 1:200 to 1:50, and most preferably from 1:150 to 1:75.

The first sample cell is used to measure the amount of components with higher concentrations, for example ethylbenzene, styrene, benzene and toluene. The second sample cell is used to measure the other components in the stream that are present at much lower levels that are measured as parts per million by weight.

The equipment used for the analyzer system must be able to withstand the high temperature of the process stream and to withstand the effects of the ethylbenzene and other components present in the stream.

In one embodiment a computer is used to determine the quantity of each component based on the reference spectra and the pressure and temperature conditions of the sample cell.

Once the analyzer measures the amounts of each component present in the sample stream, one or more of the reaction conditions may be changed to operate the dehydrogenation system more effectively. For example, the temperature may be increased to improve the conversion of ethylbenzene. The invention provides the ability to make such changes to the reaction conditions in a more timely manner and also monitor the effects of these changes in a manner that is not possible using convention manual sampling or only monitoring a purified styrene monomer stream.

The Figures show embodiments of the invention, and the features depicted in each figure will be further described to illustrate the invention. These Figures and this description are not intended to limit the invention in any way.

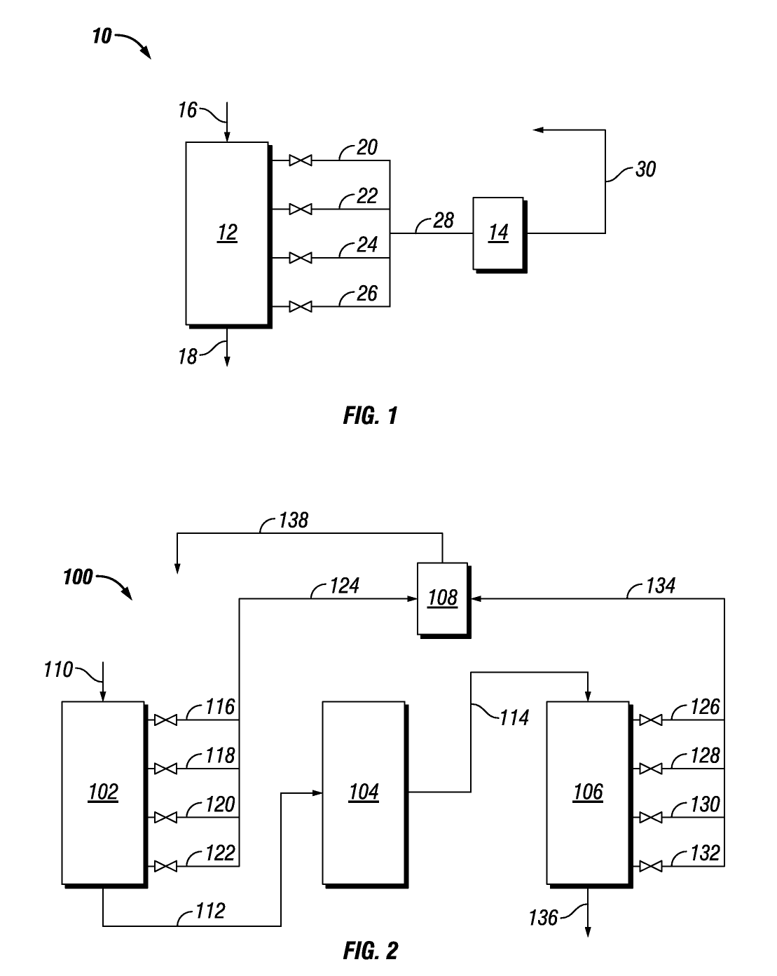

FIG. 1 depicts an embodiment of an alkylaromatic dehydrogenation system (10) comprising a dehydrogenation reactor (12) and an infrared analyzer (14). The feed to the reactor comprising ethylbenzene and steam enters the reactor through feed line (16). The dehydrogenation reactor (12) comprises a dehydrogenation catalyst and the reactor is operated at dehydrogenation reaction conditions to form styrene. The styrene and other products exit the reactor through effluent line (18). Four sample points are located on the reactor, and the appropriate valves, not all of which are shown, are operated to sequentially draw samples through sample line (26), then sample line (24), then sample line (22), and then sample line (20). The sample stream is passed through a common sample line (28) to the infrared analyzer (14). All of the sample lines are insulated and heat-traced. The sample stream is passed through one or more sample cells, and then passed through effluent line (30) that may be routed to effluent line (18) or treated separately. In another embodiment, the sample stream may be passed through the effluent line (30) and recycled to the process.

In another embodiment, there are multiple dehydrogenation reactors, typically two or three, which could all have sample points for passing samples to the infrared analyzer. In another embodiment with multiple reactors, it is possible for only one reactor to have sample points for passing samples to the infrared analyzer and in this embodiment it is preferred for the last reactor to have sample points to monitor the entire process.

FIG. 2 depicts an embodiment of a three-reactor dehydrogenation system (100) comprising two dehydrogenation reactors, one oxidative reheating reactor and an infrared analyzer. The feed comprising ethylbenzene and steam is passed through feed line (110) to the first dehydrogenation reactor (102). The dehydrogenation reactor (102) comprises a dehydrogenation catalyst and the reactor is operated at dehydrogenation reaction conditions to form styrene. The styrene and other components including hydrogen and unreacted ethylbenzene are passed through effluent line (112) to the oxidation reheating reactor (104). The oxidation reheating reactor comprises an oxidation catalyst and is operated to oxidize at least a portion of the hydrogen present. The reheated stream is then passed through effluent line (114) to the second dehydrogenation reactor (106). The dehydrogenation reactor (106) comprises a dehydrogenation catalyst and the reactor is operated at dehydrogenation reaction conditions to form additional styrene. The styrene and other products exit the reactor through effluent line (136).

Four sample points are located on the first dehydrogenation reactor (102), and the appropriate valves, not all of which are shown, are operated to sequentially draw samples through sample line (122), then sample line (120), then sample line (118), and then sample line (116). The sample stream is passed through a common sample line (124) to the infrared analyzer (108). All of the sample lines are insulated and heat-traced. The sample stream is passed through one or more sample cells, and then passed through effluent line (138) to effluent line (136) or treated separately. In another embodiment, the sample stream may be passed through the effluent line (30) and recycled to the process.

An additional four sample points are located on the second dehydrogenation reactor and samples are drawn sequentially through sample line (132), then sample line (130), then sample line (128), and then sample line (126). The sample stream is passed through a common sample line (134) to the infrared analyzer (108) and then through effluent line (138) to effluent line (136) or treated separately. In another embodiment, the sample stream may be passed through the effluent line (30) and recycled to the process.

In addition to the embodiments shown here, additional embodiments are possible. Another embodiment provides for sample points to be located on one or more of effluent lines (112), (114), and (136). A sample point located on effluent line (114) could provide more accurate monitoring of the oxidative reheating reactor including the amount of carbon monoxide and carbon dioxide produced in that reactor. In another embodiment, the sample points could be placed on one or more of the effluent lines and some or all of the sample points on the reactors could be eliminated.

Various other modifications and embodiments are possible and fall within the ability of one of ordinary skill in the art based on the disclosure provided herein. For example, the dehydrogenation process may comprise three dehydrogenation reactors and one embodiment may comprise: a first dehydrogenation reactor; a conventional reheating system using heat transfer with another stream or electrical heating; a second dehydrogenation reactor; an oxidative reheating reactor; and a third dehydrogenation reactor in that order. Another embodiment may comprise interchanging the two heating methods so that the oxidative reheating reactor is placed between the first and second dehydrogenation reactors and the conventional reheating system is placed between the second and third dehydrogenation reactors. Another embodiment comprises a system using two oxidative reheating reactors, with one in between the first and second dehydrogenation reactors and the other located between the second and third dehydrogenation reactors.

What is claimed is:

1. A method for monitoring an alkylaromatic dehydrogenation process comprising
a. drawing a sample from the process at a first sample point;
b. passing the sample through an analyzer; and
c. measuring the amount of at least one component present in the sample
wherein the sample is at least partially uncondensed.

2. A method as claimed in claim 1 wherein the analyzer comprises a source of infrared radiation and a method of detecting that radiation.

3. A method as claimed in claim 2 wherein the analyzer comprises two sample cells.

4. A method as claimed in claim 1 wherein the dehydrogenation process comprises the dehydrogenation of ethylbenzene to form styrene.

5. A method as claimed in claim 4 wherein the analyzer measures the amounts of one or more than one of the compounds selected from the group consisting of benzene, toluene, styrene, ethylbenzene, and water.

6. A method as claimed in claim 4 wherein the analyzer measures the amount of one or more than one of the compounds selected from the group consisting of alpha methylstyrene, phenylacetylene, cumene, xylene, and divinylbenzene.

7. A method as claimed in claim 4 wherein the amount of one or more of carbon monoxide or carbon dioxide is measured.

8. A method as claimed in claim 1 wherein less than 15 wt % of the sample is in a liquid state when the sample is passed through the analyzer.

9. A method as claimed in claim 1 further comprising drawing a second sample from a second sample point that is upstream of the first sample point.

10. An alkylaromatic dehydrogenation system comprising:
a. a dehydrogenation reaction zone comprising a dehydrogenation catalyst and having a plurality of sample points; and
b. a plurality of sample lines providing a flow path from the sample points to an infrared analyzer
c. wherein the sample lines are protected from heat loss sufficiently to inhibit condensation in the lines.

11. A system as claimed in claim 10 wherein the sample lines are insulated.

12. A system as claimed in claim 10 wherein the sample lines are heated.

13. A system as claimed in claim 10 wherein the infrared analyzer comprises two sample cells.

14. A system as claimed in claim 13 wherein the length of a sample cell is from 5 to 20 centimeters.

15. A system as claimed in claim 13 wherein the length of a sample cell is from 5 to 20 meters.

16. A system as claimed in claim 10 comprising an oxidative reheating reaction zone comprising a catalyst.

17. A system as claimed in claim 10 wherein the dehydrogenation catalyst also functions as an oxidation catalyst to oxidize components present in the system.

18. A method for controlling the reaction conditions of an alkylaromatic dehydrogenation system comprising
a. contacting a feed comprising an alkylaromatic compound with a dehydrogenation catalyst;
b. drawing a sample from the dehydrogenation system;
c. passing the sample through an analyzer comprising a Fourier Transform Infrared Spectrometer;
d. measuring the amount of components in the sample; and
e. adjusting the reaction conditions to improve dehydrogenation system performance
wherein the sample is at least partially uncondensed when it is passed through the analyzer.

19. A method as claimed in claim 18 further comprising contacting the feed comprising the alkylaromatic compound with an oxidation catalyst prior to contacting the feed with the dehydrogenation catalyst.

20. An apparatus for monitoring an ethylbenzene dehydrogenation process comprising
a. a plurality of sample lines that are heat-traced sufficiently to inhibit condensation in the sample lines; and
b. a Fourier Transform Infrared Spectrometer comprising two sample cells
wherein the ratio of the length of a first sample cell to the length of a second sample cell is from about 1:1000 to about 1:1.
